(19) United States
(12) Patent Application Publication
(10) Pub. No.: US 2007/0161934 A1
(43) Pub. Date: Jul. 12, 2007

(54) Foot brace for recuperation

(76) Inventor: Yuan-Hung Lan, Huantan Township (TW)

Correspondence Address: TROXELL LAW OFFICE PLLC, SUITE 1404, 5205 LEESBURG PIKE, FALLS CHURCH, VA, 22041, US

(21) Appl. No.: 11/326,468

(22) Filed: Jan. 6, 2006

(51) Int. Cl. A61F 5/00

(52) U.S. Cl. 602/23

(57) ABSTRACT

A foot brace for recuperation includes a loop, a tongue threadedly secured to the loop, two first connection units having one end threadedly secured to the tongue, two second connection units each including a recess at one end pivotably secured to a wing member at the other end of the first connection unit, a sole having either end threadedly secured to the other end of the second connection unit, and two pairs of opposite resilient members each disposed between the other end of either first connection unit and an edge of the recess. The loop is adapted to secure around the calf. The wing member is adapted to pivot about a pin when the person lifts or lowers his or her foot with one resilient member of either pair being compressed. The wing member is adapted to pivotably return to its original position after the foot touching the ground.

### BACKGROUND OF THE INVENTION

1) Field of Invention

The present invention relates to braces for recuperation and more particularly to a brace mounted on the weak foot of a person (e.g., one having a disease, an injured person, or the like) for helping the person to recuperate.

2) Related Art

The foot of a person may not be able to walk if the person has a disease or is injured. Thus, the person needs to recuperate. An endless array of recuperation equipment for such purpose is commercially available. However, the well known recuperation devices have one or more of the following problems. The knee of a person may be numb when wearing the recuperation device. Further, the person may walk with great difficulties. Furthermore, the recuperation devices are not adaptable. That is, it cannot fit different individuals with various feet. Thus, customization is required. This is very inconvenient. Thus, a need for improvement exists.

### SUMMARY OF THE INVENTION

It is therefore an object of the present invention to provide a flexible brace mounted on the foot for recuperation comprising a loop including an upper arcuate member including fastening means at both ends, and a lower member having a lower threaded hole; an inverted T-shaped tongue including an upper member including a longitudinal groove and a transverse member having a slot at either end such that the tongue and the loop are adapted to threadedly secure together by driving a fastener through the groove and the threaded hole of the loop; two first connection units each including a threaded hole at one end and a hole at the other end, and a wing member proximate the other end such that the tongue and either first connection unit are adapted to threadedly secure together by driving a fastener through the slot and the threaded hole of either first connection unit; two second connection units each including a cavity at one end, a central slit on the cavity, a recess at the other end, and a threaded hole on a center of the recess; a sole including an arcuate member and two end extensions each having a threaded hole such that either second connection unit and the sole are adapted to threadedly secure together by driving a fastener through the threaded hole of the extension and the slit, and such that either second connection unit and the either corresponding first connection unit are adapted to pivotably secure together by mounting a pivot pin through the hole of the recess and the hole of either corresponding first connection unit; and two pairs of opposite resilient members each being disposed between the other end of either first connection unit and an edge of the recess. The arcuate member is adapted to secure around the calf of a person by attaching the fastening means together, the wing member is adapted to pivot about the pivot pin when the person lifts or lowers his or her foot with one resilient member of either pair being compressed, and the wing member is adapted to return to its original position after the foot touching the ground.

In one aspect of the present invention the fastening means is a Velcro fastener.

In another aspect of the present invention there is further provided a pad on an inner surface of each of the loop, the tongue, the first connection unit, the second connection unit, and the sole.

In a further aspect of the present invention a pivot angle of the wing member about the pivot pin is in the range of 75 degrees to 105 degrees.

The above and other objects, features and advantages of the present invention will become apparent from the following detailed description taken with the accompanying drawings.

### BRIEF DESCRIPTION OF THE DRAWINGS

FIG. 1 is an exploded view of a preferred embodiment of foot brace for recuperation according to the invention;

FIG. 2 is a perspective view of the assembled foot brace in FIG 1;

FIG. 3 is an environmental view of the foot brace being worn on the foot in its lifting operation;

FIG. 3A is a broken-away plan view of a pivoting portion of the second connection unit in FIG. 3;

FIG. 4 is an environmental view of the foot brace being worn on the foot in its lowering operation; and

FIG. 4A is a broken-away plan view of a pivoting portion of the second connection unit in FIG. 4.

### DETAILED DESCRIPTION OF THE INVENTION

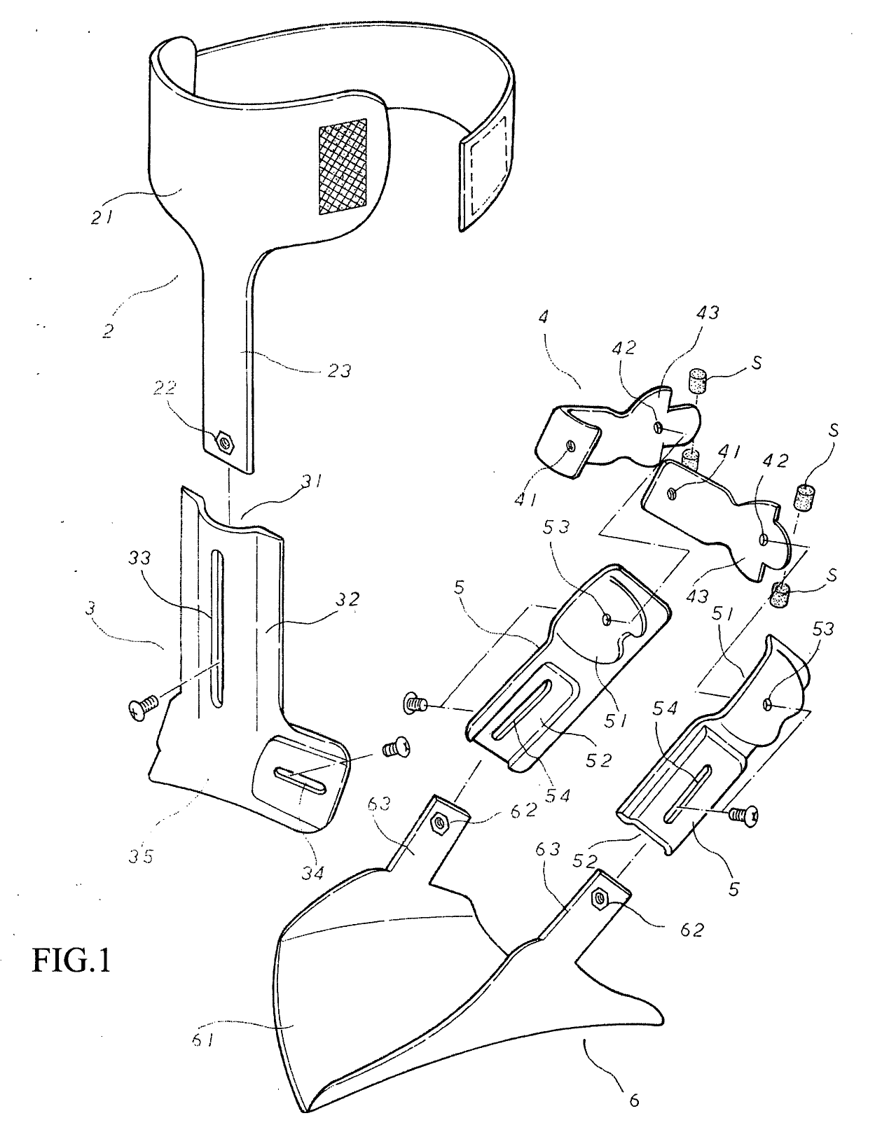

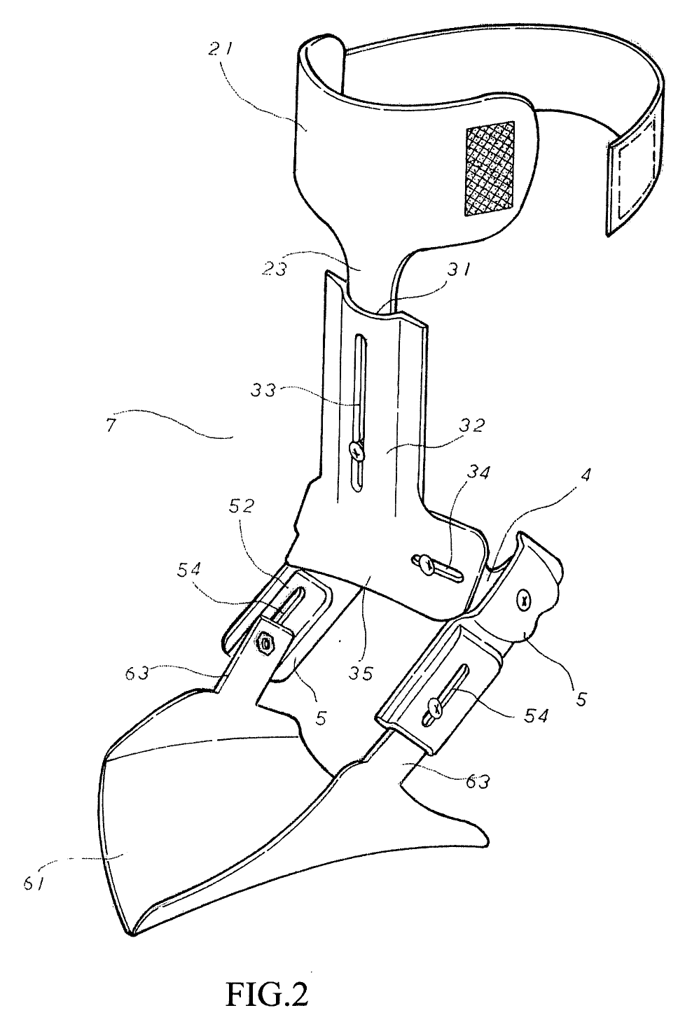

Referring to FIGS. 1, 2, and 3A, a brace 7 in accordance with a preferred embodiment of the invention is shown. The brace 7 is formed of a flexible material (e.g., plastics) and is adapted to mount on the weak foot of a person (e.g., one having a disease, an injured person, or the like) for helping the person to recuperate. The brace 7 comprises a loop 2, a tongue 3, two first connection units 4, two pairs of resilient members S, two second connection units 5, and a sole 6. Each component is discussed in detailed below.

The loop 2 comprises an upper arcuate member 21 having two mated Velcro fasteners at its both ends, and a lower member 23 having a lower threaded hole 22. The inverted T-shaped tongue 3 comprises an upper member 32 including a lengthwise, central trough 31, a lengthwise, central groove 33 along the trough 31, and a transverse member 35 having a slot 34 at its either end. The elongate first connection unit 4 comprises a threaded hole 41 at one end and a hole 42 at the other end, and a wing member 43 proximate the other hole 42. The elongate second connection unit 5 comprises a cavity 52 at one end, a central slit 54 on the cavity 52, a recess 51 at the other end, and a hole 53 on a center of the recess 51. The sole 6 comprises an arcuate member 61 and two end extensions 63 each having a threaded hole 62.

In assembly, a bolt is driven through the slit 54 into the threaded hole 62 of either extension 63 to secure either second connection unit 5 to the corresponding extension 63 (step 1). Next, performs step 1 again. A pivot pin is driven through the hole 53 into the hole 42 of either first connection unit 4 to pivotably secure either first connection unit 4 to the corresponding second connection unit 5 (step 2). Also, fit one pair of the opposite resilient members S between the other end of the first connection unit 4 and an edge of the recess 51 (step 3). Next, perform steps 2 and 3 again. A bolt is driven through one slot 34 into the threaded hole 41 of either first connection unit 4 to secure the tongue 3 to either first connection unit 4 (step 4). Next, perform step 4 again. Finally, a bolt is driven through the groove 33 into the threaded hole 22 to secure the tongue 3 to the loop 2.

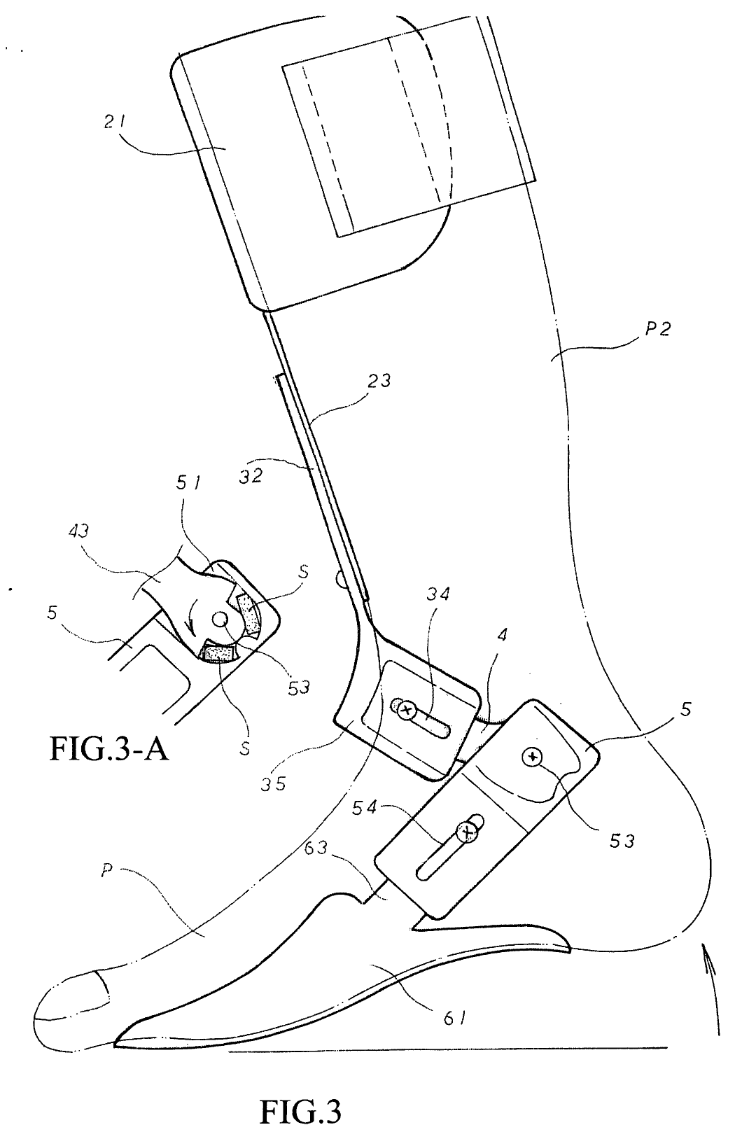

Referring to FIGS. 3 and 3A, a wearing operation of brace will be described in detailed below. A person may insert his or her foot P through the brace and suitably adjust the threaded securing to fit the foot P. Next, wrap the arcuate member 21 around the calf P2 and attach the Velcro fasteners together. The wing member 43 may pivot counterclockwise about the hole 53 when the person lifts his or her foot P. The right resilient member S is thus compressed and is able to pivotably return the wing member 43 to its original position after the foot touching the ground.

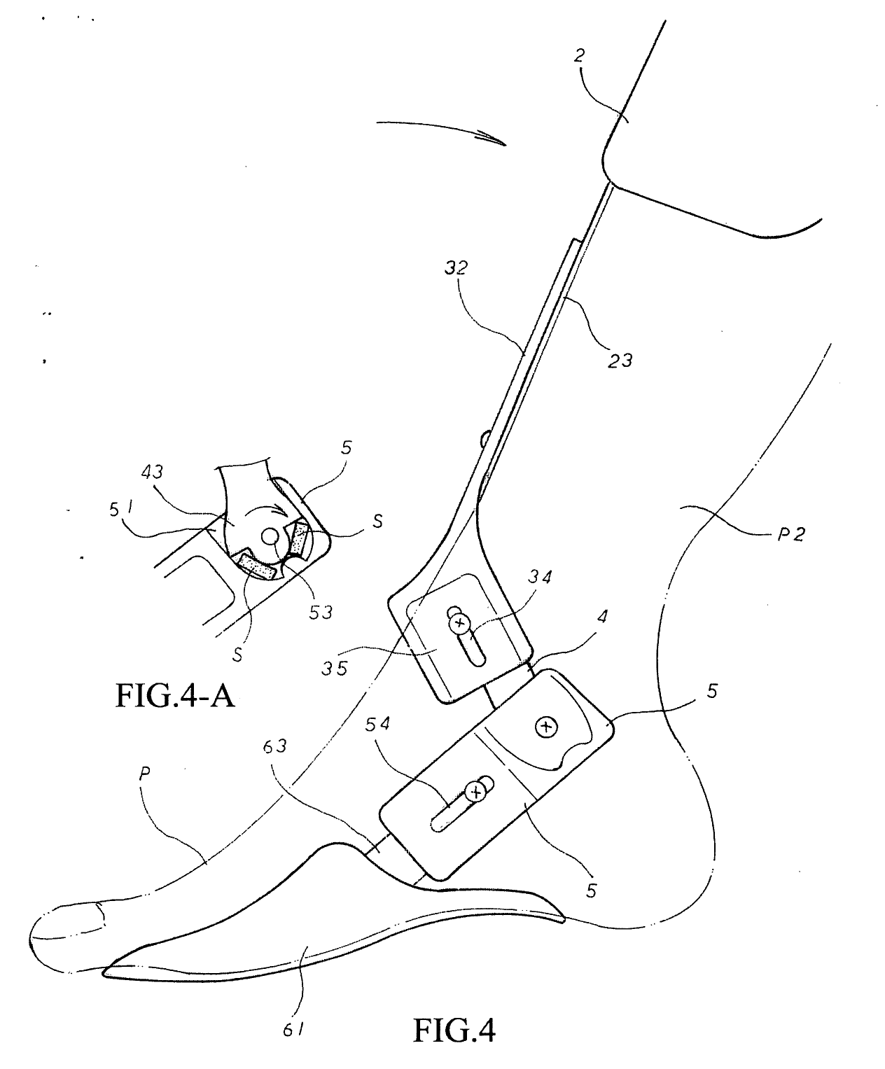

Referring to FIGS. 4 and 4A, the wing member 43 may pivot clockwise about the hole 53 when the person lowers his or her foot P. The left resilient member S is thus compressed and is able to pivotably return the wing member 43 to its original position after the foot touching the ground. Preferably, a maximum pivot angle of the wing member 43 about the hole 53 is in the range of 75 degrees to 105 degrees. More preferably, a pad is provided on an inner surface of each of the loop 2, the tongue 3, the first connection unit 4, the second connection unit 5, and the sole 6 for bringing a degree of comfort to the foot wearing the brace.

While the invention herein disclosed has been described by means of specific embodiments, numerous modifications and variations could be made thereto by those skilled in the art without departing from the scope and spirit of the invention set forth in the claims.

What is claimed is:

1. A flexible brace mounted on the foot for recuperation comprising:
a loop including an upper arcuate member including fastening means at both ends, and a lower member having a lower threaded hole;
an inverted T-shaped tongue including an upper member including a longitudinal groove and a transverse member having a slot at either end such that the tongue and the loop are adapted to threadedly secure together by driving a fastener through the groove and the threaded hole of the loop;
two first connection units each including a threaded hole at one end and a hole at the other end, and a wing member proximate the other end such that the tongue and either first connection unit are adapted to threadedly secure together by driving a fastener through the slot and the threaded hole of either first connection unit;
two second connection units each including a cavity at one end, a central slit on the cavity, a recess at the other end, and a threaded hole on a center of the recess;
a sole including an arcuate member and two end extensions each having a threaded hole such that either second connection unit and the sole are adapted to threadedly secure together by driving a fastener through the threaded hole of the extension and the slit, and such that either second connection unit and the either corresponding first connection unit are adapted to pivotably secure together by mounting a pivot pin through the hole of the recess and the hole of either corresponding first connection unit; and
two pairs of opposite resilient members each being disposed between the other end of either first connection unit and an edge of the recess;
wherein the arcuate member is adapted to secure around the calf of a person by attaching the fastening means together, the wing member is adapted to pivot about the pivot pin when the person lifts or lowers his or her foot with one resilient member of either pair being compressed, and the wing member is adapted to pivotably return to its original position after the foot touching the ground.

2. The brace of claim 1, wherein the fastening means is a Velcro fastener.

3. The brace of claim 1, further comprising a pad provided on an inner surface of each of the loop, the tongue, the first connection unit, the second connection unit, and the sole.

4. The brace of claim 1, wherein a pivot angle of the wing member about the pivot pin in the range of 75 degrees to 105 degrees.
